(19) United States
(12) Patent Application Publication
(10) Pub. No.: US 2022/0168515 A1
(43) Pub. Date: Jun. 2, 2022

(54) INHALATION MONITORING SYSTEM AND METHOD

(71) Applicant: Norton (waterford) Limited, Waterford (IE)

(72) Inventors: Mark Milton-Edwards, Macclesfield (GB); Henry Chrystyn, Eldwick (GB); Mark Steven Morrison, Basking Ridge, NJ (US); Douglas E. Weitzel, Hamilton, NJ (US)

(73) Assignee: Norton (waterford) Limited, Waterford (IE)

(21) Appl. No.: 17/675,450

(22) Filed: Feb. 18, 2022

Related U.S. Application Data
(63) Continuation-in-part of application No. 17/468,928, filed on Sep. 8, 2021.
Application No. 17/468,928 is a continuation of application No. 16/008,380, filed on Jun. 14, 2018, now Pat. No. 11,141,547.
Application No. 16/008,380 is a continuation of application No. 14/802,675, filed on Jul. 17, 2015, now Pat. No. 10,058,661.
(60) Provisional application No. 62/087,571, filed on Dec. 4, 2014.
(60) Provisional application No. 62/087,567, filed on Dec. 4, 2014.

(51) Int. Cl. A61M 15/00; A61B 5/00; A61M 16/00; G16H 20/10; G16Z 99/00; A61B 5/087; A61B 5/091

(52) U.S. Cl. CPC A61M 15/0025; A61B 5/4839; A61M 15/00; A61M 16/021; G16H 20/10; A61M 11/02; A61M 16/0003; A61B 5/0871; A61B 5/091; A61B 5/7275; A61M 16/0051; G16Z 99/00

(57) ABSTRACT

An inhalation monitoring system includes an inhaler having a medicament delivery apparatus configured to deliver medicament to a user during an inhalation of the user; inhalation monitoring apparatus, configured to, during the inhalation, gather data for determining a measure of the user's lung function and/or lung health; and a processor configured to receive the data from the inhalation monitoring apparatus and, using the data, determine a measure of the user's lung function and/or lung health.

### CROSS-REFERENCE TO RELATED APPLICATIONS

This application is a continuation-in-part of currently pending U.S. patent application Ser. No. 17/468,928, filed Sep. 8, 2021, which claims priority to U.S. patent application Ser. No. 16/008,380, filed Jun. 14, 2018, and that issued as U.S. Pat. No. 11,141,547 on Oct. 12, 2021, which claims priority to U.S. patent application Ser. No. 14/802,675, filed on Jul. 17, 2015, that issued as U.S. Pat. No. 10,058,661 on Aug. 28, 2018, which claims the benefit of Provisional U.S. Patent Application No. 62/087,567, filed Dec. 4, 2014, and U.S. Provisional Patent Application No. 62/087,571, filed December 4, each of which is incorporated by reference herein in its entirety and for all purposes.

### FIELD OF THE INVENTION

This invention is related to an inhaler, an inhalation monitoring system, and a method for monitoring an inhaler.

### BACKGROUND

Inhalers or puffers are used for delivering medication into the body via the lungs. They can be used, for example, in the treatment of asthma and chronic obstructive pulmonary disease (COPD). Types of inhalers include metered dose inhalers (MDIs), Soft Mist Inhalers (SMIs), nebulisers and dry powder inhalers (DPIs).

A tidal inhaler is a class of inhaler in which the medication is consumed in multiple successive inhalations (e.g., which may be referred to as tidal breaths) rather than a single inhalation. The patient uses their normal at rest breathing pattern without an exaggerated inhalation flow rate, also known as forced inhalation maneuver.

A spirometer is an apparatus for measuring the volume of air inspired and expired by a patient's lungs. Spirometers measure ventilation, the movement of air into and out of the lungs. From the traces, known as spirograms, output by spirometers, it is possible to identify abnormal (obstructive or restrictive) ventilation patterns. Existing spirometers use a variety of different measurement methods including pressure transducers, ultrasonic and water gauge.

Peak flow meters are used to measure peak expiratory flow (PEF), also called peak expiratory flow rate (PEFR). This is a person's maximum speed of expiration. PEF correlates with the airflow through the bronchi and thus the degree of obstruction in the airways. Peak flow readings are lower when the airways are constricted, for example due to an exacerbation of a lung condition. From changes in recorded values, patients and doctors may determine lung functionality, severity of symptoms, and treatment. Peak flow meters can also be used for diagnosis.

Spirometers and peak flow meters are generally used to monitor the lung function and/or lung health of individuals, in particular lung patients suffering from conditions such as asthma and COPD. Lung function is defined according to expiratory measures, such as PEF.

Another measure of lung function is forced expiratory volume in 1 second (FEV1). FEV1 is the volume of air that can forcibly be blown out in one second, after full inspiration. In obstructive diseases (e.g. asthma, COPD, chronic bronchitis, emphysema) FEV₁ is diminished because of increased airway resistance to expiratory flow.

Patient lung function is generally monitored during appointments with medical practitioners, periodically or in response to a recurrence or worsening of symptoms. For reasons of practicality, monitoring is typically quite infrequent during periods of apparent good health. Reactive treatment is therefore not always administered as soon as it ideally would be, and preventative treatment can be used more than necessary.

Some patients find spirometers and peak flow meters tricky to use and may need training and supervision in their use. Due to this, and for reasons of cost, most patients do not possess personal spirometers or peak flow meters.

What is needed is an improved means of monitoring lung function and/or health for patients with obstructive lung conditions.

### SUMMARY

According to a first aspect, there is provided an inhalation monitoring system comprising: an inhaler comprising medicament delivery apparatus configured to deliver medicament to a user during an inhalation of the user; inhalation monitoring apparatus configured to, during said inhalation, gather data for determining a measure of the user's lung function and/or lung health; and a processor configured to receive said data from said inhalation monitoring apparatus and, using the data, determine a measure of the user's lung function and/or lung health.

The inhaler could be a dry powder inhaler. The inhaler could be a pressurised metred dose inhaler (pMDI). The inhaler could be a wet nebuliser. The inhaler could be a tidal inhaler.

Said processor could be configured to determine said measure of the user's lung function and/or lung health by determining, from the data, peak inspiratory flow (PIF). Said processor could be configured to determine said measure of the user's lung function and/or lung health by determining, from the data, total inhaled volume. In some examples, PIF and/or total inhaled volume may be indicative of inhaler technique (e.g., good or poor inhaler technique).

The inhalation monitoring system could further comprise a user interface. Such a user interface could be configured to provide an indication of said measure of the user's lung function and/or lung health to the user. Such a user interface could be configured to provide an indication of said measure of the user's lung function and/or lung health to a caregiver. Such a user interface could be configured to provide an indication of said measure of the user's lung function and/or lung health to a medical professional.

Said indication could comprise an absolute value. Said indication could comprise a relative value. Said indication could comprise a binary health indicator. Said indication could comprise a tertiary indicator of whether the measure is above, below, or within a safe zone.

Said indication could be dependent upon data relating to the user.

The inhalation monitoring system could further comprise a transmitter. Said transmitter could be wireless.

Said transmitter could be configured to send the data to a user device for processing. Said transmitter could be configured to send the data to a user device for storage. Said transmitter could be configured to send the data to a user device for provision to the user. Said transmitter could be configured to send the data to a user device for provision to a caregiver. Said transmitter could be configured to send the data to a user device for provision to a medical professional.

Said transmitter could be configured to send the data to a server for processing. Said transmitter could be configured to send the data to a server for storage. Said transmitter could be configured to send the data to a server for provision to the user. Said transmitter could be configured to send the data to a server for provision to a caregiver. Said transmitter could be configured to send the data to a server for provision to a medical professional.

Said transmitter could be configured to send the data to a data cloud for storage.

Said transmitter could be configured to send said measure to a user device for processing. Said transmitter could be configured to send said measure to a user device for storage. Said transmitter could be configured to send said measure to a user device for provision to the user. Said transmitter could be configured to send said measure to a user device for provision to a caregiver. Said transmitter could be configured to send said measure to a user device for provision to a medical professional.

Said transmitter could be configured to send said measure to a server for processing. Said transmitter could be configured to send said measure to a server for storage. Said transmitter could be configured to send said measure to a server for provision to the user. Said transmitter could be configured to send said measure to a server for provision to a caregiver. Said transmitter could be configured to send said measure to a server for provision to a medical professional.

Said transmitter could be configured to send said measure to a data cloud for storage.

U.S. Provisional Patent App. Nos. 62/011,808 and 62/135,798, and U.S. patent application Ser. No. 14/802,675, which are each incorporated by reference herein in their entirety, describe an interface device that supports communications between a medical device and an electronic device. Such an interface could be utilized in the inhalation monitoring system that is described herein.

Said processor could be comprised in said inhaler. Said inhalation monitoring apparatus could be comprised in said inhaler. Said inhalation monitoring apparatus could be configured to be connected to said inhaler such that it is in pneumatic communication with a flow channel thereof. Said user interface could be comprised in said inhaler. Said transmitter could be comprised in said inhaler.

Said inhalation monitoring apparatus could comprise a pressure sensor. Said pressure sensor could be a microelectromechanical system (MEMS) pressure sensor. Said pressure sensor could be a barometric MEMS pressure sensor. Said pressure sensor could be a nanoelectromechanical system (NEMS) pressure sensor.

Said inhalation monitoring apparatus could be configured to gather the data by sampling a pressure differential or absolute pressure at a series of time points.

Said sampling could be periodic. Said sampling period could be approximately 50 ms. The sampling frequency could be 100 Hz, for example.

Said medicament delivery apparatus could be further configured to deliver medicament to the user during a further inhalation of the user subsequent to said inhalation. The further inhalation may be a new breath by the user using a tidal inhaler, or a continuation of the first inhalation by the user using a dry-powder inhaler, for example.

Said inhalation monitoring apparatus could be further configured to, during said further inhalation, gather further data for determining a further measure of the user's lung function and/or lung health. Said processor could be further configured to receive said further data from the inhalation monitoring apparatus. Said processor could be further configured to, using the further data, determine a further measure of the user's lung function and/or lung health. Said processor could be further configured to make a comparison of the data with the further data. Said processor could be further configured to make a comparison of the measure of the user's lung function and/or lung health with said further measure of the user's lung function and/or lung health.

The processor could be further configured to determine efficacy of usage of said inhaler using said comparison.

The processor could be further configured to predict future changes to the user's lung function and/or lung health using said comparison.

Said future changes to the user's lung function and/or lung health could comprise exacerbations of an existing respiratory condition such as asthma or chronic obstructive pulmonary disease (COPD).

The inhalation monitoring system could be configured to provide an alert to the user in response to said processor predicting one of a predetermined set of future changes to the user's lung function and/or lung health. The inhalation monitoring system could be configured to provide an alert to a caregiver in response to said processor predicting one of a predetermined set of future changes to the user's lung function and/or lung health. The inhalation monitoring system could be configured to provide an alert to a medical professional in response to said processor predicting one of a predetermined set of future changes to the user's lung function and/or lung health.

Said prediction could use data collected from subjects other than the user.

Said processor could be configured to determine said measure of the user's lung function and/or lung health using a mathematical model such as a regression model.

Said mathematical model could be of the correlation between total inhaled volume and forced expiratory volume in 1 second (FEV₁). Said mathematical model could be of the correlation between peak inspiratory flow (PIF) and forced expiratory volume in 1 second (FEV₁). Said mathematical model could be of the correlation between total inhaled volume and peak expiratory flow (PEF). Said mathematical model could be of the correlation between peak inspiratory flow (PIF) and peak expiratory flow (PEF).

For a multiple inhalation tidal inhaler or nebulizer, said mathematical model could be of the correlation between forced expiratory volume in 1 second (FEV₁) and the rate of change of the expiratory flow.

For a single inhalation dry-powder inhaler, a measurement of the user's lung function and/or lung health may be based upon a single breath by a user. For a tidal inhaler or nebulizer, the measurement may be based upon multiple breaths by the user. It is envisioned that outlying data points generated by the multiple breaths may be rejected, leaving only the good data points available for data processing.

The mathematical model could take into account biometric data for the user.

Said biometric data could comprise gender. Said biometric data could comprise age. Said biometric data could comprise height. Said biometric data could comprise weight.

The inhalation monitoring system could further comprise a user interface device operable to switch on and/or off said medicament delivery apparatus such that, when the medicament delivery apparatus is switched off, said inhaler is usable as a spirometer.

Said user interface device could comprise a mouthpiece cover of the inhaler. Said mouthpiece cover could be coupled to the medicament delivery apparatus such that a dose of medicament is made available for inhalation through a mouthpiece of the inhaler each time said cover is opened. The medicament delivery apparatus could be configured such that no further doses of medicament can be made available for inhalation through said mouthpiece until the cover has been completely closed and opened again.

The inhalation monitoring system could further comprise a placebo inhaler device. Said placebo inhaler device could comprise said inhalation monitoring apparatus. Said placebo inhaler device could be configured to be operably connected to said inhalation monitoring apparatus. Said placebo inhaler device could present substantially the same inhalation flow resistance to a user as said inhaler.

The inhalation monitoring system could comprise a battery configured to power the medicament delivery apparatus. The inhalation monitoring system could comprise a battery configured to power the inhalation monitoring apparatus. The inhalation monitoring system could comprise a battery configured to power the processor.

The inhalation monitoring system could further comprise memory configured to store the data. The inhalation monitoring system could further comprise memory configured to store said measure.

The medicament delivery apparatus of the inhalation monitoring system may comprise a medicament, and/or may be part of a kit that comprises the inhalation monitoring system and a medicament. The medicament may comprise one or more active ingredients, for example, one or more of a long-acting muscarinic antagonist (LAMA), a short-acting muscarinic antagonist (SAMA), a long-acting β₂-agonist (LABA), a short-acting β₂-agonist (SABA), and/or an inhaled corticosteroid (ICS).

According to a second aspect there is provided a method comprising: using an inhaler, delivering medicament to a user during an inhalation of the user; during said inhalation, gathering data for determining a measure of the user's lung function and/or lung health; and using the data, making a determination of a measure of the user's lung function and/or lung health.

The inhaler could be a dry powder inhaler. The inhaler could be a pressurised metred dose inhaler (pMDI). The inhaler could be a wet nebuliser. The inhaler could be a tidal inhaler.

Said determination could be made by determining, from the data, peak inspiratory flow (PIF). Said determination could be made by determining, from the data, total inhaled volume.

The method could further comprise providing an indication of said measure of the user's lung function and/or lung health to the user by means of a user interface. The method could further comprise providing an indication of said measure of the user's lung function and/or lung health to a caregiver by means of a user interface. The method could further comprise providing an indication of said measure of the user's lung function and/or lung health to a medical professional by means of a user interface.

Said indication could comprise an absolute value. Said indication could comprise a relative value. Said indication could comprise a binary health indicator. Said indication could comprise a tertiary indicator of whether the measure is above, below, or within a safe zone.

Said indication could be dependent upon data relating to the user.

The method could further comprise, by means of a transmitter, sending the data to a user device for processing. The method could further comprise, by means of a transmitter, sending the data to a user device for storage. The method could further comprise, by means of a transmitter, sending the data to a user device for provision to the user. The method could further comprise, by means of a transmitter, sending the data to a user device for provision to a caregiver. The method could further comprise, by means of a transmitter, sending the data to a user device for provision to a medical professional.

The method could further comprise, by means of a transmitter, sending the data to a server for processing. The method could further comprise, by means of a transmitter, sending the data to a server for storage. The method could further comprise, by means of a transmitter, sending the data to a server for provision to the user. The method could further comprise, by means of a transmitter, sending the data to a server for provision to a caregiver. The method could further comprise, by means of a transmitter, sending the data to a server for provision to a medical professional.

The method could further comprise, by means of a transmitter, sending the data to a data cloud for storage.

The method could further comprise, by means of a transmitter, sending said measure to a user device for processing. The method could further comprise, by means of a transmitter, sending said measure to a user device for storage. The method could further comprise, by means of a transmitter, sending said measure to a user device for provision to the user. The method could further comprise, by means of a transmitter, sending said measure to a user device for provision to a caregiver. The method could further comprise, by means of a transmitter, sending said measure to a user device for provision to a medical professional.

The method could further comprise, by means of a transmitter, sending said measure to a server for processing. The method could further comprise, by means of a transmitter, sending said measure to a server for storage. The method could further comprise, by means of a transmitter, sending said measure to a server for provision to the user. The method could further comprise, by means of a transmitter, sending said measure to a server for provision to a caregiver. The method could further comprise, by means of a transmitter, sending said measure to a server for provision to a medical professional.

The method could further comprise, by means of a transmitter, sending said measure to a data cloud for storage.

Said transmitter could be a wireless transmitter.

As noted above, according to the second aspect of the invention, there is provided a method comprising: using an inhaler, delivering medicament to a user during an inhalation of the user; during said inhalation, gathering data for determining a measure of the user's lung function and/or lung health; and using the data, making a determination of a measure of the user's lung function and/or lung health.

Said gathering could be done by said inhaler. Said determination could be made by said inhaler.

Said gathering could be done by inhalation monitoring apparatus. Said method could further comprise connecting said inhalation monitoring apparatus to the inhaler such that the inhalation monitoring apparatus is in pneumatic communication with a flow channel of the inhaler.

Said user interface could be comprised in said inhaler. Said transmitter could be comprised in said inhaler.

Said gathering could be performed by means of a pressure sensor. Said pressure sensor could be a microelectromechanical system (MEMS) pressure sensor. Said pressure sensor could be a barometric MEMS pressure sensor. Said pressure sensor could be a nanoelectromechanical system (NEMS) pressure sensor.

Said data gathering could comprise sampling a pressure differential at a series of time points. Said data gathering could also comprise sampling an absolute pressure at a series of time points.

Said sampling could be periodic.

Said sampling period could be approximately 50 ms. The sampling frequency could be 100 Hz, for example.

The method could further comprise delivering medicament to the user during a further inhalation of the user subsequent to said inhalation. The method could further comprise during said further inhalation, gathering further data for determining a further measure of the user's lung function and/or lung health. The method could further comprise using the further data, making a determination of a further measure of the user's lung function and/or lung health. The method could further comprise making a comparison of the data with the further data. The method could further comprise making a comparison of the measure of the user's lung function and/or lung health with said further measure of the user's lung function and/or lung health.

The method could further comprise determining efficacy of usage of said inhaler using said comparison.

The method could further comprise predicting future changes to the user's lung function and/or lung health using said comparison.

Said future changes to the user's lung function and/or lung health could comprise exacerbations of an existing respiratory condition such as asthma, chronic obstructive pulmonary disease (COPD), respiratory syncytial virus (RSV), Cystic Fibrosis (CF), diopathic pulmonary fibrosis (IPF), or pulmonary embolism (PE).

The method could further comprise providing an alert to the user in response to said predicting. The method could further comprise providing an alert to a caregiver in response to said predicting. The method could further comprise providing an alert to a medical professional in response to said predicting.

Said prediction could use data collected from subjects other than the user.

Said determination of said measure of the user's lung function and/or lung health could use a mathematical model. Said mathematical model could be a regression model.

Said mathematical model could be of the correlation between total inhaled volume and forced expiratory volume in 1 second (FEV₁). Said mathematical model could be of the correlation between peak inspiratory flow (PIF) and forced expiratory volume in 1 second (FEV₁). Said mathematical model could be of the correlation between total inhaled volume and peak expiratory flow (PEF). Said mathematical model could be of the correlation between peak inspiratory flow (PIF) and peak expiratory flow (PEF).

The mathematical model could take into account biometric data for the user.

Said biometric data could comprise gender. Said biometric data could comprise age. Said biometric data could comprise height. Said biometric data could comprise weight.

The method could further comprise: switching off a medicament delivery function of the inhaler; and using the inhaler as a spirometer.

For a single inhalation dry-powder inhaler, for example, switching off said medicament delivery function could comprise opening a mouthpiece cover of the inhaler. Said cover could be configured such that a dose of medicament is made available for inhalation through a mouthpiece of the inhaler each time the cover is opened. The inhaler could be configured such that no further doses of medicament can be made available for inhalation through the mouthpiece until the cover has been completely closed and opened again.

The method could further comprise using a placebo inhaler device.

Said gathering could be performed by inhalation monitoring apparatus. Said placebo inhaler device could comprise said inhalation monitoring apparatus. Said placebo inhaler device could be configured to be operably connected to said inhalation monitoring apparatus.

Said placebo inhaler device could present substantially the same inhalation flow resistance to a user as said inhaler. For a multiple inhalation tidal inhaler or a wet nebulizer, which may have a lower inhalation flow resistance than a dry-powder inhaler, it may be advantageous to use a special non-drug cartridge having a defined inhalation flow resistance. The non-drug cartridge could electronically identify itself to the inhaler by the same means that is used to identify the drug within a cartridge, e.g., via an electrically erasable programmable read-only memory.

The method could further comprise storing the data and/or said measure in memory.

An inhalation monitoring system may include an inhaler and an inhalation monitoring apparatus. The inhaler may include medicament, a mouthpiece, a mouthpiece cover, and/or a medicament delivery apparatus. The medicament delivery apparatus may be configured to deliver medicament to a user during an inhalation of the user. In some examples, the medicament delivery apparatus may comprise a bellows, hooper, and a dosing cup, such as those described herein through reference, or other systems, such as those used to advance blister strips of dry powder medicament, systems used to open capsules of dry powder medicament, etc.

The inhalation monitoring apparatus may include a processor, memory, power source, a sensor, and a communication circuit (e.g., a wireless communication circuit that, for example, may be configured to communicate accordingly to Bluetooth Low Energy). The sensor may include a pressure sensor (e.g., barometric or differential pressure sensor), an acoustic sensor, an optical sensor, etc.). The inhalation monitoring apparatus may be configured to, during said inhalation by the user, gather data for determining a measure of the user's lung function and/or lung health. In some examples, the measure of the user's lung function and/or lung health may (e.g., itself) comprise the total inhaled volume of said inhalation. In some examples, the inhalation monitoring apparatus may be comprised within (e.g., integrally formed within) the inhaler. In other examples, the inhalation monitoring apparatus may be removably attached to and detached from the inhaler, such that the inhalation monitoring apparatus may be moved between inhalers. Further, in even other examples, the inhalation monitoring apparatus may be a standalone unit that is configured for use with, not at attachment to, the inhaler.

The inhalation monitoring system may also include a processor, such as one or more processors that resides at an external device, like a smartphone, tablet, personal computer, and/or server. The processor (e.g., of the external device) may be configured to receive said data from said inhalation monitoring apparatus and, using the data, determine the measure of the user's lung function and/or lung health. In some examples, the processor may be configured to determine said measure of the user's lung function and/or lung health by determining, from the data, the total inhaled volume of said inhalation. As such, in some instances, the inhalation monitoring apparatus may offload some processing to the processor of the external device.

The processor is further configured to provide, via a user interface (e.g., a user interface of the external device), an indication of the total inhaled volume to at least one of the user, a caregiver, or a medical professional. For instance, the processor may generate a graphical user interface (GUI) via the user interface of the external device, where the GUI comprises the indication of the total inhaler volume. The processor may be further configured to provide, via the user interface, a numerical value for the total inhaled volume (e.g., in liters). The processor may further be configured to provide, via the user interface, an indication of whether the numerical value of the total inhaled volume is within a range (e.g., a healthy range and/or a range indication of good or poor inhaler technique), where, for instance, the boundaries of such a range could again depend on biometric data stored for the particular user. In some examples, the indication provided via the user interface may provide an indication of whether the inhalation was too weak for effective drug administration.

The processor (e.g., of the external device) may determine a total inhaled volume for each of a plurality of inhalations. The processor may receive the total inhaled volume for each of the plurality of inhalations, and/or the processor may receive data, that was gathered during each of the plurality of inhalations, and determine, based on the received data, the measure of the user's lung function and/or lung health of each of the plurality of inhalation. The plurality of inhalations may be a plurality of inhalations from a single user (e.g., using the inhaler or multiple inhalers), or may be a plurality of inhalation from a plurality of different users (e.g., using different inhalers). The processor may be configured to simultaneously provide, via the user interface (e.g., in a single GUI), an indication of the total inhaled volume for a plurality of inhalations (e.g., as illustrated in the Figures). As such, the user (e.g., of the external device) may be able to examine, simultaneously, the total inhaled volume for the plurality of inhalations. Further, in some examples, the processor may also generate, via the user interface (e.g., within the GUI), a trend line across the total inhaled volume for the plurality of inhalations. The trend line may be a line of best fit across the total inhaled volume for the plurality of inhalations.

Alternatively or additionally, the processor may be configured to determine whether the total inhaled volume is within a range (e.g., a healthy range and/or a range indication of good or poor inhaler technique), and based on the determination, generate, via the user interface, an indication of whether the user inhalation was the result of good or poor inhaler technique.

The processor may categorize each of the plurality of inhalations as a good inhalation, a low inhalation, an exhalation, and/or a possible air vent block (e.g., an excessive inhalation that is indicative of the air vent being block or obstructed by the user during the inhalation) based on a peak flow rate for each of the plurality of inhalations. For instance, the processor and/or the inhalation monitoring apparatus may be configured to determine the peak flow rate for each inhalation based on said data that is gathered during the inhalation. The processor may provide, via the user interface, an indication for each of the plurality of inhalations of whether the inhalation is categorized as a good inhalation, a low inhalation, an exhalation, and/or a possible air vent block.

An inhalation monitoring system may include an inhaler and one or more processors. The inhaler may include comprising a medicament delivery apparatus configured to deliver medicament to a user during an inhalation of the user. In some examples, the inhaler may include medicament, a mouthpiece, and a mouthpiece cover. A first processor may be configured to, during said inhalation, gather data for determining a measure of the user's lung function and/or lung health. In some examples, the first processor may be part of the inhaler or an add-on device that attaches to the inhaler. A second processor may be configured to determine, using the data, the measure of the user's lung function and/or lung health, wherein the processor is configured to determine said measure of the user's lung function and/or lung health by determining, from the data, total inhaled volume. A third processor may be configured to receive the total inhaled volume of said inhalation, and provide, via a user interface, an indication of the total inhaled volume to at least one of the user, a caregiver, or a medical professional.

Any combination of the first, second, and third processors may be the same processor. For example, the processing may be performed at the same or different locations. For instance, in some examples, the first and second processors may be the same processor. For example, the first and second processor may be a single processor that is part of an inhalation monitoring apparatus. In some examples, the second processor may be different from the first processor. For example, the first processor may be part of the inhalation monitoring apparatus and the second processor may be part of an external device, such as a smartphone, tablet, personal computer, and/or server.

In some examples, the second and third processors may be the same processor. For example, the second and third processors may be a single processor that is part of an external device. In some examples, the second processor may be different from the third processor. For example, the second processor may be part of a first external device, such as a smartphone, tablet, personal computer, and/or server, while the third processor is part of a second, different external device. Finally, in some examples, the first, second, and third processors may be the same processor, such as a processor of the inhalation monitoring apparatus.

The third processor may be configured to provide, via the user interface, a numerical value for the total inhaled volume. The third processor may be configured to provide, via the user interface, an indication of whether the numerical value of the total inhaled volume is within a range. In some examples, the indication may further provide an indication of whether the inhalation was too weak for effective drug administration.

The third processor may be configured to simultaneously provide, via the user interface, an indication of the total inhaled volume for a plurality of inhalations (e.g., the plurality of inhalations may be for a single user and/or for a plurality of different users). In some examples, a processor (e.g., the second processor and/or the third processor) may be configured to generate, via the user interface, a trend line across the total inhaled volume for the plurality of inhalations. Further, in some examples, the processor (e.g., the second processor and/or the third processor) may be configured to categorize each of the plurality of inhalations as a good inhalation or a low inhalation based on a peak flow rate for each of the plurality of inhalations, and configured to provide, via the user interface, an indication for each of the plurality of inhalations of whether the inhalation is categorized as a good inhalation or a low inhalation based on the peak flow rate for each inhalation. For instance, the processor (e.g., the second processor and/or the third processor) may be configured to categorize each of the plurality of inhalations as a good inhalation, a low inhalation, an exhalation, or a possibly air vent block based on the peak flow rate for each of the plurality of inhalations, and configured to provide, via the user interface, an indication for each of the plurality of inhalations of whether the inhalation is categorized as a good inhalation, a low inhalation, an exhalation, or a possibly air vent block based on the peak flow rate for each inhalation. The processor (e.g., the second processor and/or the third processor) may be to provide, via the user interface, the categorization of each of the plurality of inhalations in one or more rows, wherein each row is dedicated a single category, wherein the rows are configured above or below a chart that comprises a plotted total inhaled volume for each inhalation. In some examples, each plot within the chart represents a single good inhalation of the plurality of inhalation and a respective time for the inhalation.

The third processor may be to provide simultaneously, via the user interface, a chart that comprises a plot of the total inhaled volume of a plurality of inhalations, and an indication of the categorization of each of the plurality of inhalations as a good inhalation, a low inhalation, an exhalation, and/or a possible air vent block. The categorization of each of the plurality of inhalation may be based on a peak flow rate for each inhalation.

### BRIEF DESCRIPTION OF THE DRAWING FIGURES

Aspects of the present invention will now be described by way of example with reference to the accompanying figures. In the figures:

FIG. 1a illustrates an example correlation between PEF and the maximum flow measured during inhalation;

FIG. 1b illustrates an example correlation between FEV₁ and total inhaled volume;

FIG. 2 schematically illustrates an example inhalation monitoring system;

FIG. 3 is a flowchart of an example inhalation monitoring method;

FIG. 4a illustrates an example inhalation volume chart that includes the inhalation volume for a plurality of inhalations; and

FIG. 4b illustrates another example inhalation volume chart that includes the inhalation volume for a plurality of inhalations.

### DETAILED DESCRIPTION

The following description is presented to enable any person skilled in the art to make and use the system, and is provided in the context of a particular application. Various modifications to the disclosed embodiments will be readily apparent to those skilled in the art.

The general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

Many lung patients are prescribed inhalers so that they or a caregiver can administer medicament to them as a routine preventative measure, to ease an exacerbation, or both. Such patients and caregivers are trained in the use of these inhalers and become very familiar with them. It is therefore proposed to monitor patients' lung function using their inhalers. Monitoring lung health while administering medication reduces the time and effort required from patients, caregivers and medical professionals to manage lung conditions.

This has not been previously considered since, as explained above, lung function is generally assessed using expiratory measures and dry powder inhalers, for example, are not generally designed to permit exhalation. In some cases, for example some dry powder inhalers, exhalation into inhalers can impair their function (e.g., if moisture from an exhalation causes powdered medicament to form clumps, making even administration more difficult).

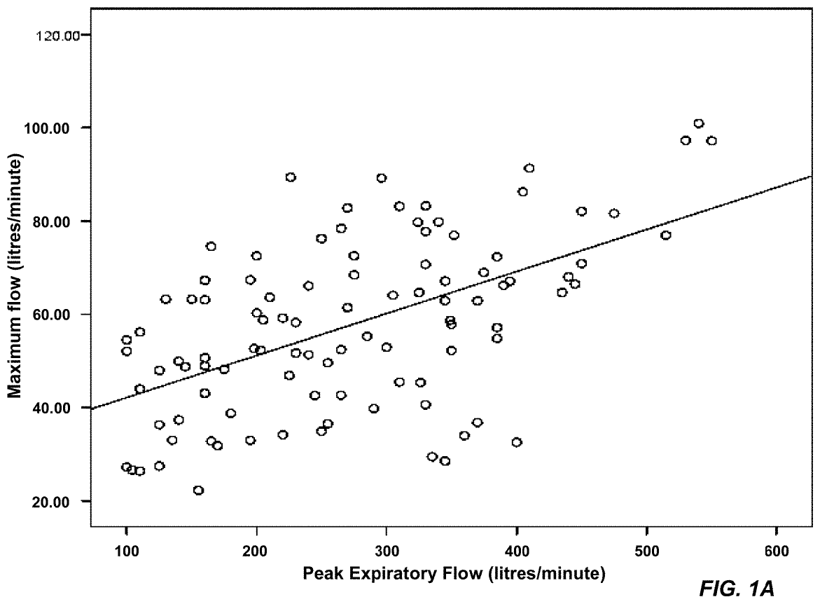

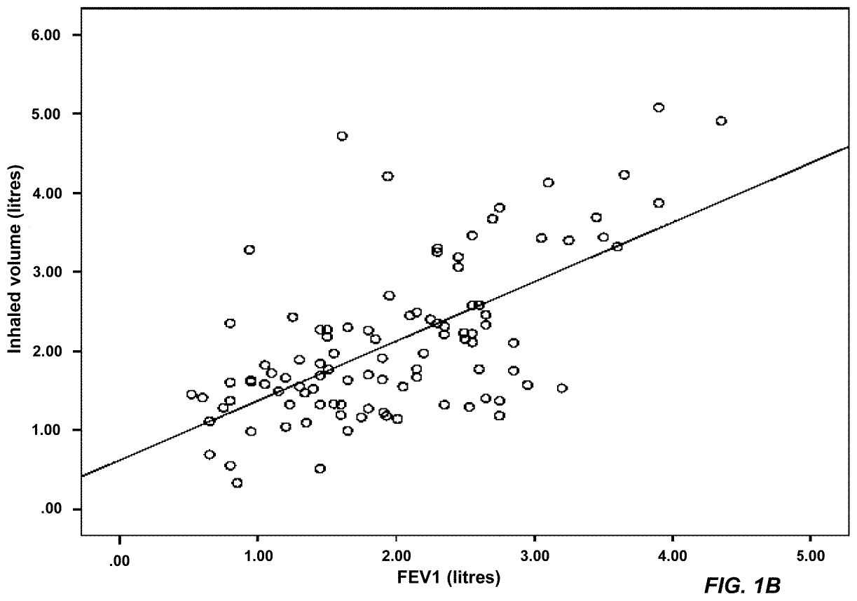

However, the applicant has established that there are correlations between some expiratory measures of lung function and some inspiratory measures. For example, see FIG. 1a, showing a correlation between PEF and the maximum flow measured during inhalation (peak inspiratory flow, PIF), and FIG. 1b, showing a correlation between FEV1 and total inhaled volume. Regression lines and their equations are indicated on the plots, where:

x₁=gender (male=0; female=1)

x₂=age/years

x₃=height/cm

x₄=weight/kg

x₅=PEF/l·min⁻¹

x₆=FEV1/l·min⁻¹

y₁=inhaled volume/I

y₂=PIF/I

It is therefore proposed to process inhalation data gathered while administering medicament with an inhaler in order to determine lung function and/or health.

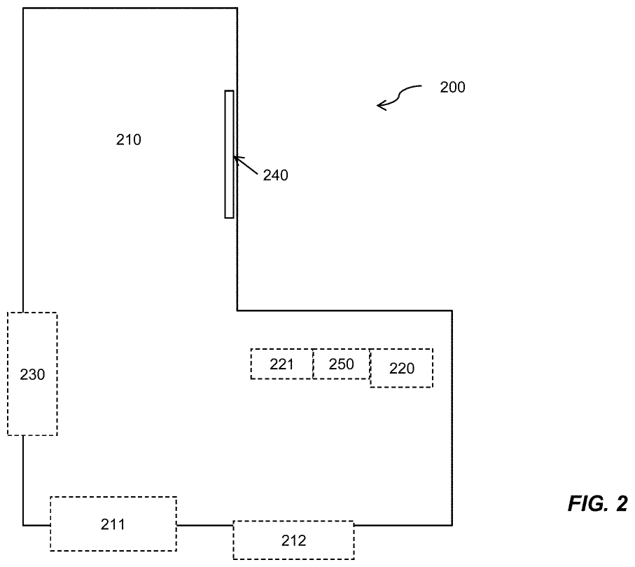

FIG. 2 schematically illustrates an example inhalation monitoring system 200. An inhaler 210 comprises medicament delivery apparatus 211. This could for example be as per the dry powder inhalers described in any of PCT patent application publication numbers WO 01/97889, WO 02/00281, WO 2005/034833 or WO 2011/054527, which are incorporated in their entirety herein. Inhalation monitoring systems could also comprise other types of inhalers/nebulisers, for example, pressurised metred dose inhalers (pMDIs) or wet nebulisers. The inhalers could require forced inspiratory manoeuvres or only tidal breathing.

Inhalation monitoring apparatus 220 may also be comprised in the inhaler as shown, or may be comprised in a separate unit connected to it. The inhalation monitoring apparatus may comprise a sensor, such as a pressure sensor (e.g., barometric or differential), an acoustic sensor, an optical sensor, and/or the like. For instance, the inhalation monitoring apparatus could for example comprise a miniature (e.g., microelectromechanical, MEMS, or nanoelectromechanical, NEMS) pressure sensor as described in any of US patent application No. 62/043,126 to Morrison, Ser. No. 62/043,120 to Morrison, and 62/043,114 to Morrison, which are incorporated in their entirety herein. Other suitable arrangements could be envisaged. For those making use of a pressure sensor, said sensor should be in pneumatic communication with an airflow channel of the inhaler through which the user inhales.

A processor 230 communicates with the inhalation monitoring apparatus in order to process data collected by the inhalation monitoring apparatus to determine a measure of the user's lung function and/or health. The processor 230 could be comprised in the inhaler as shown, or if the inhalation monitoring apparatus is comprised in a separate accessory unit, the processor could also be comprised in said accessory unit. If the inhalation monitoring apparatus is equipped with a wired or wireless transmitter 221 (e.g., a Bluetooth Low Energy chipset), the processor could be in a separate device, for example a user device such as a smartphone, tablet, smart laptop, PC, and/or server. If the inhalation monitoring apparatus is equipped with a transmitter capable of communicating with a network such as the internet, the processing could be done remotely, for example at a medical professional's PC or on a health service, inhaler manufacturer or cloud server. For example, any of the abovementioned devices or servers could also be used for data storage. Processor 230 may be made up of multiple processors in any of the abovementioned locations. For example, some basic processing may be done on board the inhaler, while more detailed analysis is offloaded to a remote device or server.

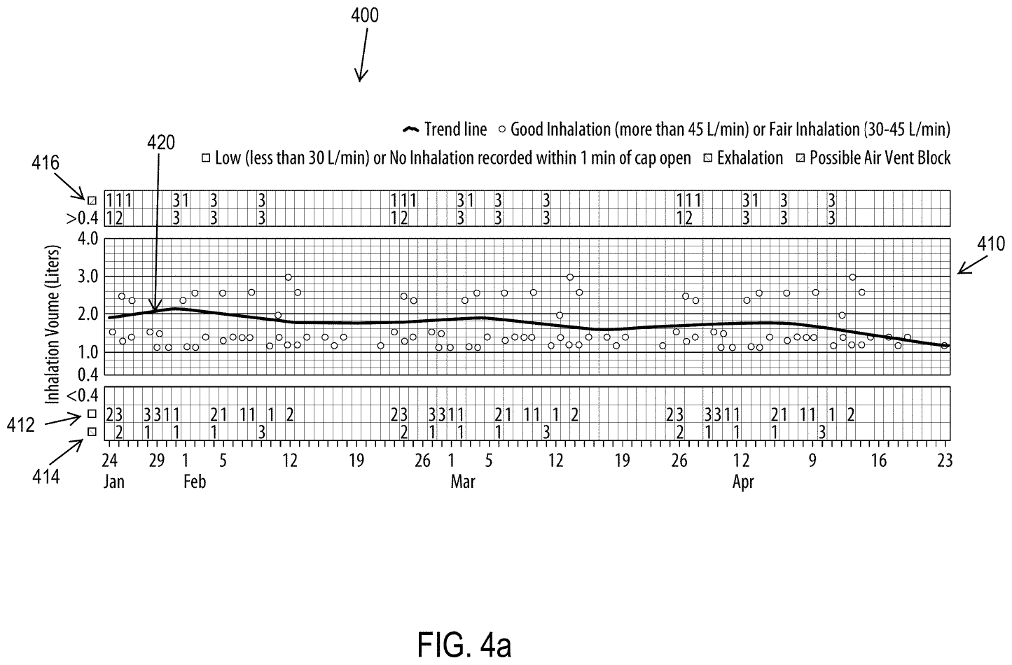

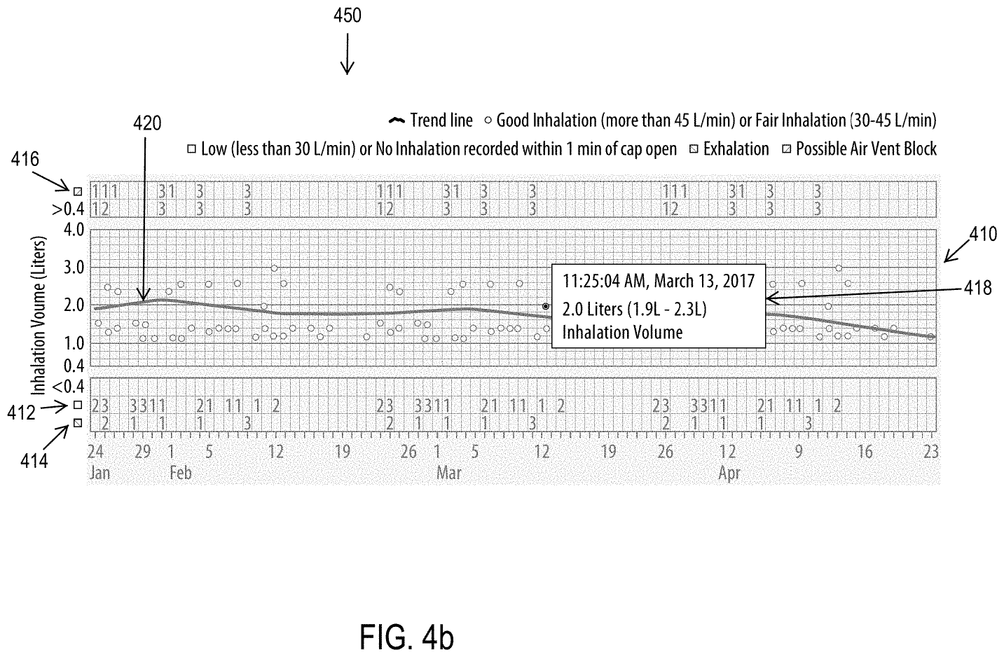

The inhaler may comprise a user interface 240 for providing information relating to use of the inhaler and/or determined lung function and/or lung health. This could, for example, be a screen, indicator light, indicator buzzer, speaker, traditional dose counter tape, vibrating alert etc. or any combination of these or similar. Alternatively or additionally, such information could be provided via one or more user interfaces of a user device (e.g., an external device) of the patient or a caregiver or medical professional. For example, the user device may comprise a display, and the processor of the user device may be configured to generate one or more user interfaces (e.g., graphical user interfaces (GUIs)) that provide an indication of the measure of the user's lung function and/or lung health, such as inhalation volume, PIF, PEF, FEV1, etc. (e.g., as shown in FIGS. 1a, 1b, 4a, and/or 4b).

The system could also comprise a memory 250 for storing the collected data, calculation results and computer code instructions for execution by the processor. As with the processor, the memory could be located in the inhaler or an external device (e.g., smartphone, tablet, PC, and/or server). The memory 250 may comprise a computer-readable storage media or machine-readable storage media that maintains computer-executable instructions for performing one or more procedures as described herein. For example, the memory 250 may comprise computer-executable instructions or machine-readable instructions that include one or more portions of the procedures described herein. The inhalation monitoring apparatus and/or the remote device may access the instructions from memory 250 for being executed to cause the inhalation monitoring apparatus and/or the remote device to operate as described herein, or to operate one or more other devices as described herein. The memory 250 may comprise computer-executable instructions for executing configuration software.

The electronic component of the inhaler could be powered by a battery 212 so that the inhaler can be portable. For instance, the inhalation monitoring apparatus may comprise the battery 212, which may be replaceable and/or rechargeable.

The inhaler could further comprise switching means for putting the medicament delivery apparatus in or out of operation. When the medicament delivery apparatus is not functioning, the inhaler can be used as a spirometer. As one example, electronic switching means could be provided if the medicament delivery apparatus is under electronic (e.g., push-button) control. As another example, PCT patent application publication number WO 2005/034833, which is incorporated by reference herein in its entirety, describes a mechanism for a metered dose dry powder inhaler in which a metering cup measures out a dose of medicament from a hopper and is moved to a dosing position by action of a yoke linked to a mouthpiece cover. Thus, opening the mouthpiece cover primes the inhaler for use and once a dose has been inhaled, further dosing is not possible until the cover has been closed and opened again. Using such an inhaler with the inhalation monitoring apparatus proposed herein, a patient could take their dose of medicament and, before closing the mouthpiece cover, make one or more further inhalations through the mouthpiece for the purposes of further data collection. This allows greater volumes of data to be collected without risking the patient over-dosing. As yet another example, a spirometer cartridge could be connected to a replaceable cartridge tidal inhaler, and a patient could make one or more further inhalations through the spirometer cartridge for the purpose of further data collection.

Alternatively or additionally, the inhaler described above could be provided in a kit with a placebo or dummy inhaler which has a similar flow resistance to the real inhaler, but which either does not comprise medicament delivery apparatus, is empty or is loaded with a placebo substance such as lactose. The placebo inhaler could comprise similar inhalation monitoring apparatus to that described above, or could be connectable to such apparatus.

If inhaler 210 were a wet nebulizer, for example, then all of the electronic components could be located in a module that is removably connected to the inhalation port in order to protect the electronics from exposure to fluid. The module could be configured to be connected to different wet nebulizers of varying shape and size. The module could include a flow channel having a defined inhalation flow resistance that is higher than the inhalation flow resistance of the wet nebulizer alone (e.g., without the module).

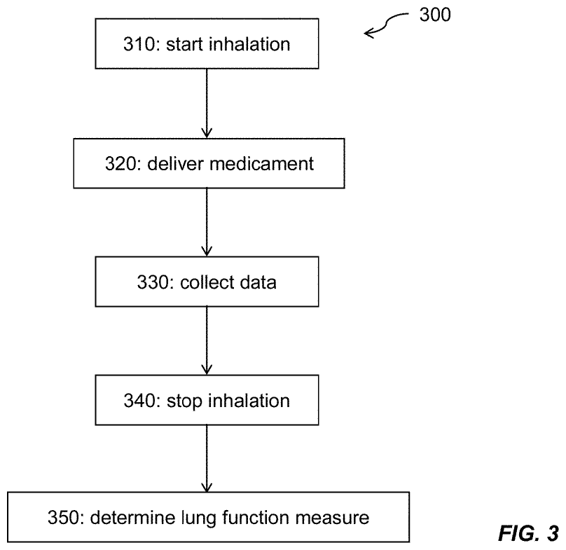

FIG. 3 is a flowchart of an example inhalation monitoring method 300. At 310, inhalation (through an inhaler) commences. At 320, medicament is delivered via the inhaler. At 330, data concerning said inhalation is collected. At 340, inhalation ends. At 350, the data is processed to make a determination of a measure of lung function and/or lung health. The order of steps 320 and 330 could be reversed or they could be carried out partially or fully in parallel. Step 350 could occur before, during or after 340 and before, after, or fully or partially in parallel with 320. Further, in some examples, step 330 may be performed by the inhalation monitoring apparatus, while 350 may be performed by the inhalation monitoring apparatus and/or the external device. If 350 is performed at the external device, the method 300 may include a step of sending the data to the external device by the inhalation monitoring apparatus. Further, the method may include providing, at a user interface (e.g., a user interface of the external device), an indication of the total inhaled volume to at least one of the user, a caregiver, or a medical professional. For instance, the processor may generate a graphical user interface (GUI) via the user interface of the external device, where the GUI comprises the indication of the total inhaler volume, PIF, PEF, and/or FEV1. The processor may be further configured to provide, via the user interface, a numerical value for the total inhaled volume (e.g., in liters). The processor may further be configured to provide, via the user interface, an indication of whether the numerical value of the total inhaled volume is within a range (e.g., a healthy range), where, for instance, the boundaries of such a range could again depend on biometric data stored for the particular user.

The data could also be used for adherence monitoring by a medical practitioner, e.g., to ensure that the inhaler is being used properly by the user.

The processing could comprise use of a mathematical model such as the regression models illustrated in FIG. 1.

Method 300 could be repeated each time the inhaler is used, which could for example be daily. Data gathered from multiple uses of the inhaler and/or determinations made from the data could be stored and compared to provide an indication of the progression of a condition over time. This information could be used to determine efficacy of the current treatment regime and inform any changes which may be required. The processor may also be capable of using the data and/or determinations to predict future changes in lung function and/or lung health. This prediction could be based on date (e.g., only on data) collected from the patient in question, and/or could incorporate data collected from other patients too. For example, data from users of many inhalers as described above could be collated and used to identify patterns in inhalation data changes preceding exacerbations of particular lung conditions. The processing logic could thus be self-learning. If a particular patient's data is then seen to match the beginning of such a pattern, they or their caregiver or medical practitioner could be alerted so that any required changes to a treatment regime (for example, increased dosage, additional medications or therapies) can be made to help avoid an exacerbation.

The data collected by the inhalation monitoring apparatus could be, for example, a time series of measurements. For example, the data collected by the inhalation monitoring apparatus could be, for example, a time series of pressure differential measurements or absolute pressure measurements. Measurements could be made periodically, for example every 10 ms, 50 ms or 100 ms over e.g. 2, 5 or 10 seconds. Data collection may be reset between uses of the inhalation monitoring apparatus.

The user interface could provide a numerical value, for example of measured PIF, calculated total inhaled volume, calculated PEF, calculated FEV1 or a fraction or percentage of one of these relative to an ideal value for the particular patient (e.g., said ideal value could be chosen based on biometric data such as age, gender, height, weight etc.). Alternatively or additionally, it could provide a binary indicator as to whether or not the measured value is within a healthy range, or a tertiary indicator as to whether the measured value is below, above or within a healthy range. Boundaries of such a healthy range could again depend on biometric data stored for the particular patient. The user interface could alternatively or additionally be used to indicate number of doses taken or number of doses remaining in a disposable inhaler, refillable hopper or disposable cartridge. Another alternative or additional indication could be whether the inhaler has been used correctly, for example so that the patient or a caregiver or medical professional is alerted to missed doses, inhalations that are too short or weak for effective drug administration, or that medication has otherwise been taken incorrectly, and/or receives confirmation that medication has been taken correctly.

FIG. 4a illustrates an example inhalation volume chart 400 that includes the inhalation volume for a plurality of inhalations. FIG. 4b illustrates another example inhalation volume chart 450 that includes the inhalation volume for a plurality of inhalations.

As described herein, the processor (e.g., of the external device) may determine a total inhaled volume for each of a plurality of inhalations. The processor may receive the total inhaled volume for each of the plurality of inhalations (e.g., from the inhalation monitoring system), and/or the processor may receive data, that was gathered during each of the plurality of inhalations, and determine, based on the received data, the measure of the user's lung function and/or lung health of each of the plurality of inhalation. The plurality of inhalations may be a plurality of inhalations from a single user (e.g., using a single inhaler or multiple inhalers, each comprising an inhalation monitoring system), or may be a plurality of inhalation from a plurality of different users (e.g., using different inhalers comprising an inhalation monitoring system).

The processor may be configured to simultaneously provide, via the user interface (e.g., in a single GUI), an indication of the total inhaled volume for a plurality of inhalations (e.g., as illustrated in FIG. 1b, 4a, and/or 4b). The user interface may be provided via a display of the external device. As such, the user (e.g., of the external device) may be able to examine, simultaneously, the total inhaled volume for the plurality of inhalations. For example, referring to FIGS. 4a and 4b, the user interface may provide a chart 400 and/or 450 that includes a plurality of plots within a chart 410 of the user interface, where each plot is associated with an inhalation of the plurality of inhalation, and where each plot indicates an inhalation volume (Liters) of the inhalation and a time (e.g., day and/or time) of the inhalation. Further, in some instances, the processor may be configured to generate a trend line 420 across the plots of the total inhaled volume for the plurality of inhalations within the chart 410. The trend line 420 may, for example, provide a visual illustration of a user's inhalation volume of the plurality of inhalations over time. The trend line 420 may be a line of best fit across the total inhaled volume for the plurality of inhalations.

In some examples, such as that shown in FIG. 4b, the processor may be configured to receive a selection of a plot for an inhalation, and in response to the selection, the processor may be configured to generate (e.g., simultaneously in the same user interface) an indication of the numerical value of the inhaled volume of the inhalation, potentially along with a more exact time of the inhalation and/or an inhalation volume range of the inhalation as shown by box 418. The selection may be received via the user interface (e.g., via a touch sensitive display of the external device). The range may, for example, indicate the accuracy of the inhalation volume determined by the processor, since for example, the inhalation volume may be calculated from another metric, such as peak flow rate, which itself may be measured by the inhalation monitoring system (e.g., based on measurements received from the sensor).

In some examples, the processor (e.g., of the external device) may be configured to categorize each of the plurality of inhalations as a good inhalation, a low inhalation, an exhalation, or a possible air vent block. In such examples, the user interface may also comprise an indication of the category of each inhalation, which for example, may be provided in one or more rows that are dedicated to each category above and/or below the plotted inhalation volume for each inhalation (e.g., as shown in FIGS. 4a and 4b). For instance, the user interface may comprise a row 412 that indicates that an inhalation is a low inhalation, a row 414 that indicates that an inhalation is an exhalation, and/or a row 416 that indicates whether an inhalation is a possibly air vent block. The rows 412, 414, and 416 may be located above or below the chart 410. Further, in some examples, the chart 410 may be reserved for inhalations that are categorized as good inhalations (e.g., as shown in FIGS. 4a and 4b), for instance, such that each plot within the chart 410 represents a single good inhalation at a particular time. However, in other examples, the chart 410 may include a plot for every inhalation, regardless of its categorization.

In some examples, the processor may categorize each of the plurality of inhalations as a good inhalation, a low inhalation, an exhalation, and/or a possible air vent block based on a peak flow rate for each of the plurality of inhalations. For instance, the processor and/or the inhalation monitoring apparatus may be configured to determine the peak flow rate for each inhalation based on said data that is gathered during the inhalation. The processor may provide, via the user interface, an indication for each of the plurality of inhalations of whether the inhalation is categorized as a good inhalation, a low inhalation, an exhalation, and/or a possible air vent block based on the peak flow rate for each inhalation. As such, in some examples, the processor may be configured to provide a chart 210 that includes a plot of the inhalation volume of a plurality of inhalations, and simultaneously in the same user interface, also provide a categorization of each of the plurality of inhalations as a good inhalation, a low inhalation, an exhalation, and/or a possible air vent block based on the peak flow rate for each inhalation. Finally, and as an example, the processor may categorize an inhalation as a good inhalation if the peak flow rate is between 30-200 L/min, a low inhalation if the peak flow rate is below 30 L/min, an excessive flow rate if the peak flow rate is above 200 L/min, and an exhalation if the measurements from the inhalation monitoring system indicates a positive pressure change.

The inhaler is preferably directed to the treatment of respiratory disorders such as asthma and/or COPD. A range of classes of medicaments have been developed to treat respiratory disorders, and each class has differing targets and effects.

Bronchodilators are employed to dilate the bronchi and bronchioles, decreasing resistance in the airways, thereby increasing the airflow to the lungs. Bronchodilators may be short-acting or long-acting. Typically, short-acting bronchodilators provide a rapid relief from acute bronchoconstriction, whereas long-acting bronchodilators help control and prevent longer-term symptoms.

Different classes of bronchodilators target different receptors in the airways. Two commonly used classes are anticholinergics and β₂-agonists.

Anticholinergics (or “antimuscarinics”) block the neurotransmitter acetylcholine by selectively blocking its receptor in nerve cells. On topical application, anticholinergics act predominantly on the M3 muscarinic receptors located in the airways to produce smooth muscle relaxation, thus producing a bronchodilatory effect. Preferred examples of long-acting muscarinic antagonists (LAMAs) include tiotropium (bromide), oxitropium (bromide), aclidinium (bromide), ipratropium (bromide) glycopyrronium (bromide), oxybutynin (hydrochloride or hydrobromide), tolterodine (tartrate), trospium (chloride), solifenacin (succinate), fesoterodine (fumarate) and darifenacin (hydrobromide). In each case, particularly preferred salt/ester forms are indicated in parentheses. Preferred examples of short-acting muscarinic antagonists (SAMAs) include tropicamide and cyclopentolate.

β₂-Adrenergic agonists (or “β₂-agonists”) act upon the β₂-adrenoceptors which induces smooth muscle relaxation, resulting in dilation of the bronchial passages. Preferred long-acting β₂-agonists (LABAs) include formoterol (fumarate), salmeterol (xinafoate), indacaterol (maleate), bambuterol (hydrochloride), clenbuterol (hydrochloride), olodaterol (hydrochloride), carmoterol (hydrochloride), tulobuterol (hydrochloride) and vilanterol (triphenylacetate). Examples of short-acting β₂-agonists (SABAs) include salbutamol (sulfate), terbutaline (sulfate), pirbuterol (acetate), metaproterenol (sulfate), and albuterol. In each case, particularly preferred salt/ester forms are indicated in parentheses.

Another class of medicaments employed in the treatment of respiratory disorders are inhaled corticosteroids (ICSs). ICS are steroid hormones used in the long-term control of respiratory disorders. They function by reducing the airway inflammation. Preferred examples include budesonide, beclomethasone (dipropionate), fluticasone (propionate or furoate), mometasone (furoate), ciclesonide and dexamethasone (sodium). In each case, particularly preferred salt/ester forms are indicated in parentheses.

The active ingredients may be administered in combination, and both combination therapies and combination products have been proposed. Examples of combination treatments and products disclosed in the art are set out in WO 2004/019985, WO 2007/071313, WO 2008/102128 and WO 2011/069197. The active ingredients can be a combination of a LAMA, LABA and an ICS. They may be a double combination of a LAMA and a LABA, a LAMA and an ICS, a LABA and an ICS, and/or the like. They may also be a combination of a LAMA, a LABA, and an ICS.

Example combinations are:

oxybutynin (hydrochloride or hydrobromide) and formoterol (fumarate) darifenacin (hydrobromide) and formoterol (fumarate)

oxybutynin (hydrochloride or hydrobromide), formoterol (fumarate) and beclomethasone (dipropionate)

darifenacin (hydrobromide), formoterol (fumarate) and beclomethasone (dipropionate)

oxybutynin (hydrochloride or hydrobromide) and salmeterol (xinafoate)

darifenacin (hydrobromide) and salmeterol (xinafoate)

oxybutynin (hydrochloride or hydrobromide), salmeterol (xinafoate) and fluticasone (propionate)

darifenacin (hydrobromide), salmeterol (xinafoate) and fluticasone (propionate)

glycopyrronium (bromide) and indacaterol (maleate)

glycopyrronium (bromide) and formoterol (fumarate)

tiotropium (bromide) and formoterol (fumarate)

tiotropium (bromide) and carmoterol (hydrochloride)

tiotropium (bromide) and olodaterol (hydrochloride)

tiotropium (bromide) and indacaterol (maleate)

budesonide and formoterol (fumarate)

A number of approaches have been taken in formulating these classes of active ingredients for delivery by inhalation, such as via a dry powder inhaler (DPI), a pressurised metered dose inhaler (pMDI), or a nebuliser.

The API of the medicament should penetrate deep into the lung in order to reach their site of action. Therefore, the APIs are micronized to obtain particles having the required size, typically a mass medium aerodynamic diameter (MMAD) of 1-5 μm.

The medicament may be delivered as pure drug, but more appropriately, it is preferred that medicaments are delivered together with excipients (carriers) which are suitable for inhalation. Suitable excipients include organic excipients such as polysaccharides (e.g. starch, cellulose and the like), lactose, glucose, mannitol, amino acids, and maltodextrins, and inorganic excipients such as calcium carbonate or sodium chloride. Lactose is a preferred excipient.

Particles of powdered medicament and/or excipient may be produced by conventional techniques, for example by micronisation, milling or sieving.

Additionally, medicament and/or excipient powders may be engineered with particular densities, size ranges, or characteristics. Particles may comprise active agents, surfactants, wall forming materials, or other components considered desirable by those of ordinary skill.

The medicament may be incorporated into the reservoir of an inhaler or into a canister to be placed inside of an inhaler. Alternatively, the medicament may be presented separately to the inhaler, for example in a blister strip of unit doses or capsules which can form a kit of parts with the inhaler.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

What is claimed is:

1. An inhalation monitoring system comprising:
an inhaler comprising a medicament delivery apparatus configured to deliver medicament to a user during an inhalation of the user;
an inhalation monitoring apparatus configured to, during said inhalation, gather data for determining a measure of the user's lung function and/or lung health; and
a processor configured to receive said data from said inhalation monitoring apparatus and, using the data, determine the measure of the user's lung function and/or lung health, wherein the processor is configured to determine said measure of the user's lung function and/or lung health by determining, from the data, total inhaled volume.

2. The inhalation monitoring system of claim 1, wherein the processor is further configured to provide, via a user interface, an indication of the total inhaled volume to at least one of the user, a caregiver, or a medical professional.

3. The inhalation monitoring system of claim 2, wherein the processor is further configured to provide, via the user interface, a numerical value for the total inhaled volume.

4. The inhalation monitoring system of claim 3, wherein the processor is further configured to provide, via the user interface, an indication of whether the numerical value of the total inhaled volume is within a range.

5. The inhalation monitoring system of claim 2, wherein the processor is further configured to simultaneously provide, via the user interface, an indication of the total inhaled volume for a plurality of inhalations.

6. The inhalation monitoring system of claim 5, wherein the processor is further configured to generate, via the user interface, a trend line across the total inhaled volume for the plurality of inhalations.

7. The inhalation monitoring system of claim 5, wherein the processor is further configured to:
categorize each of the plurality of inhalations as a good inhalation or a low inhalation based on a peak flow rate for each of the plurality of inhalations; and
provide, via the user interface, an indication for each of the plurality of inhalations of whether the inhalation is categorized as a good inhalation or a low inhalation based on the peak flow rate for each inhalation.

8. The inhalation monitoring system of claim 5, wherein the processor is further configured to:
categorize each of the plurality of inhalations as a good inhalation, a low inhalation, an exhalation, or a possibly air vent block based on the peak flow rate for each of the plurality of inhalations; and
provide, via the user interface, an indication for each of the plurality of inhalations of whether the inhalation is categorized as a good inhalation, a low inhalation, an exhalation, or a possibly air vent block based on the peak flow rate for each inhalation.

9. The inhalation monitoring system of claim 8, wherein the processor is configured to provide, via the user interface, the categorization of each of the plurality of inhalations in one or more rows, wherein each row is dedicated a single category, wherein the rows are configured above or below a chart that comprises a plotted total inhaled volume for each inhalation.

10. The inhalation monitoring system of claim 9, wherein each plot within the chart represents a single good inhalation of the plurality of inhalation and a respective time for the inhalation.

11. The inhalation monitoring system of claim 2, wherein the processor is configured to provide simultaneously, via the user interface, a chart that comprises a plot of the total inhaled volume of a plurality of inhalations, and an indication of the categorization of each of the plurality of inhalations as a good inhalation, a low inhalation, an exhalation, and/or a possible air vent block

12. The inhalation monitoring system of claim 11, wherein the categorization of each of the plurality of inhalation is based on a peak flow rate for each inhalation.

13. The inhalation monitoring system of claim 1, wherein the processor is configured to:
receive data, that was gathered during a plurality of inhalations, for determining a measure of the user's lung function and/or lung health;
determine a total inhaled volume for each of the plurality of inhalations; and
simultaneously provide, via the user interface, an indication of the total inhaled volume for each of the plurality of inhalations.

14. The inhalation monitoring system of claim 13, wherein each of the plurality of inhalations occurred during a use of the inhaler by the user.

15. The inhalation monitoring system of claim 1, wherein said inhalation monitoring apparatus is comprised in said inhaler, and said processor is comprises in an external device.

16. A method comprising:
delivering medicament to a user during a plurality of inhalations using an inhaler;
gathering data, during each of the plurality of inhalations, for determining a measure of the user's lung function and/or lung health;
determining total inhaled volume for each of the plurality of inhalations;
simultaneously providing, via a user interface, an indication of the total inhaled volume for each of the plurality of inhalation; and
generating, via the user interface, a trend line across the total inhaled volume for the plurality of inhalations.

17. An inhalation monitoring system comprising:
an inhaler comprising a medicament delivery apparatus configured to deliver medicament to a user during an inhalation of the user;
a first processor configured to, during said inhalation, gather data for determining a measure of the user's lung function and/or lung health;
a second processor configured to determine, using the data, the measure of the user's lung function and/or lung health, wherein the processor is configured to determine said measure of the user's lung function and/or lung health by determining, from the data, total inhaled volume; and
a third processor configured to receive the total inhaled volume of said inhalation, and provide, via a user interface, an indication of the total inhaled volume to at least one of the user, a caregiver, or a medical professional.

18. The inhalation monitoring system of claim 17, wherein the first and second processors are a single processor comprised with an inhalation monitoring apparatus that is comprised within or attachable to the inhaler.

19. The inhalation monitoring system of claim 17, wherein the second and third processors are a single processor comprised with an external device.

20. The inhalation monitoring system of claim 17, wherein the third processor is further configured to:
simultaneously provide, via the user interface, an indication of the total inhaled volume for a plurality of inhalations, wherein the plurality of inhalation are associated with the user; and
generate, via the user interface, a trend line across the total inhaled volume for the plurality of inhalations.
